(19) United States
(12) Patent Application Publication
(10) Pub. No.: US 2008/0274192 A1
(43) Pub. Date: Nov. 6, 2008

(54) Pharmaceutical Compositions Comprising an Amorphous Form of a Vegf-R-Inhibitor

(75) Inventors: Dwayne Thomas Friesen, Bend, OR (US); Douglas Alan Lorenz, Bend, OR (US); Scott Wendell Smith, San Diego, CA (US)

Correspondence Address: PFIZER INC, 10555 SCIENCE CENTER DRIVE, SAN DIEGO, CA, 92121, US

(73) Assignee: PFIZER INC., New York, NY (US)

(21) Appl. No.: 11/914,289

(22) PCT Filed: May 8, 2006
(86) PCT No.: PCT/IB06/01295

§ 371 Date: Jul. 7, 2008

Related U.S. Application Data
(60) Provisional application No. 60/682,928, filed on May 19, 2005.

(51) Int. Cl. A61K 9/00; A61K 31/4439; A61P 35/00

(52) U.S. Cl. 424/488; 514/339

(57) ABSTRACT

A pharmaceutical composition comprising the compound 6-[2-(methylcarbamoyl)phenylsulfanyl]-3-E-[2-(pyridin-2-yl)ethenyl]indazole, or a pharmaceutically acceptable salt or solvate thereof, in an amorphous form.

This application claims priority to U.S. Patent Application No. 60/682,928, filed May 19, 2005, which is hereby incorporated by reference.

### FIELD OF THE INVENTION

The present invention relates to pharmaceutical compositions comprising the compound 6-[2-(methylcarbamoyl)phenylsufanyl]-3-E-[2-(pyridin-2-yl)etheny]indazole in an amorphous form. The compositions of the present invention are useful for treating diseases or conditions mediated by VEGF-R, such as, for example, disease states associated with abnormal cell growth such as cancer.

### BACKGROUND

The invention relates to pharmaceutical compositions comprising an inhibitor of vascular endothelial growth factor receptor (VEGF-R), a member of the growth factor receptor tyrosine kinase family of protein kinases. The compound 6-[2-(methylcarbamoyl)phenylsulfanyl]-3-E-[2-(pyridin-2-yl)ethenyl]indazole (hereinafter referred to as “Compound A”) is an inhibitor of VEGF-R that may be used for the treatment of disease states associated with abnormal cell growth. Compound A is disclosed as Example 33(a) in U.S. Pat. No. 6,534,524, the disclosure of which is incorporated herein by reference. Compound A belongs to a class of compounds known as indazole compounds, which inhibit the activity of certain protein kinases. By inhibiting tyrosine kinase signal transduction, Compound A inhibits unwanted cell proliferation. Compound A can be used to treat cancer and other disease states associated with unwanted cellular proliferation, such as diabetic retinopathy, neovascular glaucoma, rheumatoid arthritis, and psoriasis.

Compound A can exist in several different crystalline forms, as described in U.S. Provisional Application 60/624,665, filed on Nov. 2, 2004, which is incorporated herein by reference. Compound A in the crystalline form referred to as polymorphic Form IV has a pKa of about 4.2, and a solubility that is dependent on the pH of the solution, with the solubility being higher at low pH than at high pH. Compound A has a solubility of about 4 μg/mL in model fasted duodenal solution (an aqueous solution comprising 20 mM Na₂HPO₄, 47 mM KH₂PO₄, 87 mM NaCl, and 0.2 mM KCl, adjusted to pH 6.5 with NaOH, in which is additionally present 7.3 mM sodium taurocholic acid and 1.4 mM of 1-palmitoyl-2-oleyl-sn-glycero-3-phosphocholine) at a temperature of 37° C. This pH-dependent solubility combined with a low in vivo rate of absorption results in low oral bioavailability, as well as significant subject-to-subject pharmacokinetic variability, for crystalline Compound A. Accordingly, there is a need to improve the bioavailability and reduce pharmacokinetic variability of Compound A relative to its crystalline form.

### SUMMARY

The invention provides a pharmaceutical composition comprising Compound A, wherein at least a portion of Compound A is amorphous. Amorphous Compound A has improved solubility relative to crystalline Compound A, and when dosed orally provides improved bioavailability relative to crystalline Compound A.

One aspect of the present invention provides amorphous Compound A, or a pharmaceutically acceptable salt or solvate thereof.

Another aspect of the present invention provides pharmaceutical compositions comprising amorphous Compound A, or a pharmaceutically acceptable salt or solvate thereof.

In still another aspect of the present invention are provided pharmaceutical compositions comprising Compound A, or a pharmaceutically acceptable salt or solvate thereof, wherein at least 5 wt % of the total amount of Compound A present is in an amorphous form. Further, pharmaceutical compositions are provided comprising Compound A, or a pharmaceutically acceptable salt or solvate thereof, wherein at least 10 wt %, or at least 15 wt % or at least 20 wt %, or at least 30 wt %, or at least 40 wt %, or at least 50 wt %, or at least 60 wt %, or at least 70 wt %, or at least 80 wt %, or at least 90 wt %, or at least 95 wt % of the total amount of Compound A present is in an amorphous form.

In one aspect, the pharmaceutical compositions comprise (1) amorphous Compound A, or a pharmaceutically acceptable salt or solvate thereof, and (2) a matrix. In a further aspect said composition and said matrix are in the form of a solid amorphous dispersion. In a further aspect said solid amorphous dispersion is substantially homogeneous.

In another aspect of the present invention are provided pharmaceutical compositions comprising Compound A, or a pharmaceutically acceptable salt or solvate thereof, and a matrix, wherein at least 5 wt % of the total amount of Compound A present is in an amorphous form. Further, pharmaceutical compositions are provided comprising Compound A, or a pharmaceutically acceptable salt or solvate thereof, and a matrix, wherein at least 10 wt %, or at least 15 wt % or at least 20 wt %, or at least 30 wt %, or at least 40 wt %, or at least 50 wt %, or at least 60 wt %, or at least 70 wt %, or at least 80 wt %, or at least 90 wt %, or at least 95 wt % of the total amount of Compound A present is in an amorphous form.

In still another aspect are provided pharmaceutical compositions, comprising Compound A, or a pharmaceutically acceptable salt or solvate thereof, and a matrix, wherein said matrix comprises at least one of an ionizable cellulosic polymer, a nonionizable cellulosic polymer, and a noncellulosic polymer.

In still further aspects, the at least one ionizable cellulosic polymer is selected from hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, hydroxypropyl methyl cellulose acetate phthalate, cellulose acetate terephthalate and cellulose acetate isophthalate, and mixtures thereof.

Still further are provided such compositions wherein the at least one nonionizable, cellulosic polymer is selected from hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose, and mixtures thereof.

Other aspects provide such compositions wherein said at least one non-cellulosic polymer is selected from carboxylic acid functionalized polymethacrylates, carboxylic acid functionalized polyacrylates, amine-functionalized polyacrylates, amine-fuctionalized polymethacrylates, proteins, carboxylic acid functionalized starches, vinyl polymers and copolymers having at least one substituent selected from the group consisting of hydroxyl, alkylacyloxy, and cyclicamido, vinyl copolymers of at least one hydrophilic, hydroxyl-containing repeat unit and at least one hydrophobic, alkyl- or aryl- containing repeat unit, polyvinyl alcohols that have at least a portion of their repeat units in the unhydrolyzed form, polyvinyl alcohol polyvinyl acetate copolymers, polyethylene glycol polypropylene glycol copolymers, polyvinyl pyrrolidone, polyethylene polyvinyl alcohol copolymers, polyoxyethylene-polyoxypropylene block copolymers and mixtures thereof.

In another embodiment, the invention provides a pharmaceutical composition comprising Compound A and a matrix, wherein at least a portion of Compound A is in an amorphous form, and wherein the composition, when administered to an in vivo or in vitro aqueous use environment, provides at least one of (a) a maximum dissolved concentration of Compound A in the use environment that is at least 1.25-fold that provided by a control composition; and (b) a concentration of Compound A in the use environment versus time area under the curve (AUC) for any period of at least 90 minutes between the time of introduction into the use environment and 270 minutes following introduction to the use environment that is at least 1.25-fold that of the control composition. The control composition consists essentially of an equivalent quantity of Compound A in polymorphic Form IV alone. In particular embodiments, the maximum dissolved concentration of Compound A in the use environment is at least 1.5-fold, at least 2-fold, at least 4-fold, at least 8-fold, at least 10-fold, at least 15-fold, or at least 20-fold that provided by a control composition. In further embodiments the concentration of Compound A in the use environment versus time area under the curve (AUC) for any period of at least 90 minutes between the time of introduction into the use environment and 270 minutes following introduction to the use environment is at least 1.5-fold, at least 2-fold, at least 4-fold, at least 8-fold, at least 10-fold, or at least 15-fold that of the control composition.

The present invention further relates to pharmaceutical compositions comprising Compound A, or a pharmaceutically acceptable salt or solvate thereof, and a matrix, wherein at least a portion of said compound is in an amorphous form, and wherein when administered to an in vivo use environment, said composition provides at least one of: a) a dose-normalized AUC value of said compound in the blood plasma or serum that is at least 5-fold that provided by a control composition; and b) a dose-normalized Cₘₐₓ value of said compound in the blood plasma or serum that is at least 5-fold that provided by said control composition; wherein said control composition is administered under similar conditions as said pharmaceutical composition and consists essentially of Compound A in polymorphic Form IV. In a further embodiment, said composition provides a dose-normalized Cₘₐₓ value that is at least 6-fold, at least 7-fold, at least 8-fold, at least 9-fold, or at least 10-fold that provided by said control composition. In a further embodiment, said composition provides a dose-normalized AUC value that is at least 6-fold, at least 7-fold, at least 8-fold, at least 9-fold, at least 10-fold, at least 11-fold, or at least 12-fold that provided by said control composition. In further embodiments, said in vivo use environment is the GI tract of an animal, such as a dog or a human. In a further embodiment, said pharmaceutical composition and said control composition are both administered under fasted conditions.

In a further embodiment is a pharmaceutical composition comprising Compound A, or a pharmaceutically acceptable salt or solvate thereof, and a matrix, wherein at least a portion of said compound is in an amorphous form, and wherein when administered to an in vivo use environment in multiple subjects, said composition provides at least one of: a) an AUC coefficient of variation that is less than 95% of the AUC coefficient of variation provided by said control composition; and b) a Cₘₐₓ coefficient of variation that is less than 95% of the Cₘₐₓ coefficient of variation provided by said control composition; wherein said control composition is administered under similar conditions as said pharmaceutical composition and consists essentially of Compound A in polymorphic Form IV. In a further embodiment, said composition provides an AUC coefficient of variation that is less than 90%, less than 80%, less than 70%, or less than 65% of the AUC coefficient of variation provided by said control composition. In a further embodiment, said composition provides a Cₘₐₓ coefficient of variation that is less than 90%, less than 80%, less than 70%, or less than 65% of the Cₘₐₓ coefficient of variation provided by said control composition. In further embodiments, said in vivo use environment is the GI tract of an animal, such as a dog or a human. In a further embodiment, the number of subjects is at least four. In a further embodiment, said pharmaceutical composition and said control composition are both administered under fasted conditions.

The present invention further relates to a method of reducing the AUC coefficient of variation of Compound A to less than 95%, less than 90%, less than 80%, less than 70%, or less than 50% of the AUC coefficient of variation provided by a control composition of Compound A that is administered under similar conditions and consists essentially of Compound A in polymorphic Form IV, wherein said method comprises administering any of the pharmaceutical compositions of Compound A as described herein. The present invention further relates to a method of reducing the Cₘₐₓ coefficient of variation of Compound A to less than 95%, less than 90%, less than 80%, less than 70%, or less than 50% of the Cₘₐₓ coefficient of variation provided by a control composition of Compound A that is administered under similar conditions and consists essentially of Compound A in polymorphic Form IV, wherein said method comprises administering any of the pharmaceutical compositions of Compound A as described herein.

The present invention further relates to a composition comprising a solid amorphous dispersion of the compound 6-[2-(methylcarbamoyl)phenylsulfanyl]-3-E-[2-(pyridin-2-yl)ethenyl]indazole or a pharmaceutically acceptable salt or solvate thereof, and a matrix, wherein said solid amorphous dispersion reduces pharmacokinetic variability of said compound in vivo.

The invention also relates to methods of reducing abnormal cell growth in a mammal in need thereof, comprising the step of administering to said mammal any of the pharmaceutical compositions described herein. In one embodiment, said abnormal cell growth in cancerous.

The invention further relates to a use of any of the compositions described herein in the manufacture of a medicament for the treatment of abnormal cell growth in a mammal.

In a further embodiment of the present invention is a process for preparing a pharmaceutical composition comprising: dissolving a compound in a spray solution comprising a solvent; and rapidly evaporating said solvent from said spray solution to afford an amorphous form of said compound; wherein said compound is 6-[2-(methylcarbamoyl)phenylsulfanyl]-3-E-[2-(pyridin-2-yl)ethenyl]indazole, or a pharmaceutically acceptable form thereof. In one particular embodiment of the process described above, said spray solution further comprises a matrix. In certain other embodiments said matrix comprises at least one polymer selected from an ionizable cellulosic polymer, a nonionizable cellulosic polymer, and a noncellulosic polymer. In a further embodiment, said matrix is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate and cellulose acetate isophthalate. In a further embodiment of the present invention, said solvent is selected from the group consisting of methanol, acetone, and mixtures of methanol and acetone.

As used herein, a “use environment” can be either the in vivo environment, such as the GI tract of an animal, particularly a human, or the in vitro environment of a test solution, such as phosphate buffered saline (PBS) solution or Model Fasted Duodenal (MFD) solution.

As used here in, the term “at least a portion of Compound A is in an amorphous form” means that at least 5 wt %, preferably at least 10 wt % of the total amount of Compound A in the composition is in an amorphous form.

The term “equivalent quantity” as used herein refers to molar quantities of Compound A, measured as the theoretical number of moles of parent compound, 6-[2-(methylcarbamoyl)phenylsulfanyl]-3-E-[2-(pyridin-2-yl)ethenyl]indazole, present in a given composition. For example, for a given amount of a composition comprising a salt or solvate of 6-[2-(methylcarbamoyl)phenylsulfanyl]-3-E-[2-(pyridin-2-yl)ethenyl]indazole, an equivalent quantity of polymorphic Form IV of Compound A would be calculated by determining the theoretical number of moles of 6-[2-(methylcarbamoyl)phenylsulfanyl]-3-E-[2-(pyridin-2-yl)ethenyl]indazole present in the composition and using an amount of Form IV of compound A that would afford the same theoretical number of moles of compound A.

The term “Compound A,” unless stated otherwise, is meant to refer to the compound 6-[2-(methylcarbamoyl)phenylsufanyl]-3-E-[2-(pyridin-2-yl)ethenyl]indazole, or a pharmaceutically acceptable salt or solvate thereof. By “pharmaceutically acceptable form” is meant any pharmaceutically acceptable derivative or variation, including stereoisomers, stereoisomer mixtures, enantiomers, solvates, hydrates, isomorphs, polymorphs, pseudomorphs, neutral forms, salt forms, and prodrugs.

The term “crystalline,” as used herein, means a particular solid form of a compound of the invention that exhibits long-range order in three dimensions. Material that is crystalline may be characterized by techniques known in the art such as powder x-ray diffraction (PXRD) crystallography, solid state NMR, or thermal techniques such as differential scanning calorimetry (DSC).

The term “amorphous,” as used herein, means a particular solid form of a compound of the invention that has essentially no order in three dimensions. The term “amorphous” is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC).

The terms “administration,” “administering,” “dosage,” and “dosing,” as used herein refer to the delivery of a compound, or a pharmaceutically acceptable salt or solvate thereof, or of a pharmaceutical composition containing the compound, or a pharmaceutically acceptable salt or solvate thereof, to a mammal such that the compound is absorbed into the serum or plasma of the mammal.

“Dose-normalized” refers to the dose-adjusted value of a particular parameter, wherein dose can refer to: 1) the amount of drug administered per body weight of the subject receiving the drug (e.g. 8 mg/kg); or 2) the total amount of drug administered to the subject (e.g. 20 mg). The dose-normalized value of a particular parameter is calculated by dividing the value of the parameter by the dose. For example, if the dose of drug administered to the subject is 8 mg/kg, and the AUC value is 2.0 μg hr/mL, then the dose-normalized AUC value is (2.0 μg hr/mL)/8 mg/kg=0.25 μg hr/mL/mg/kg. Further for example, if the dose of drug administered to the subject is 10 mg, and the Cₘₐₓ value is 1.0 μg/mL then the dose-normalized Cₘₐₓ value is 1.0 μg/mL/10 mg=0.1 μg/mL/mg. It should be understood that although “dose-normalized” refers to normalization by either the amount of drug per body weight of the subject, or by total amount of drug, when comparing dose-normalized values between a composition of the present invention and a control composition as described herein, the dose-normalized values should be calculated in the same manner (i.e. using either amount of drug per body weight, or total amount of drug, for both test and control dose-normalized values).

The term “administered under similar conditions” refers to the in vivo administration conditions. Such conditions include the fed or fasted state and the group of subjects involved. For example, similar administration conditions means administering to the same group of subjects that are in the same fed or fasted state, wherein an appropriate washout period (e.g. one week) exists between dosing of the test and control compositions.

As used herein, the term “fasted” means the subject has not consumed any food or liquid for at least 2 hours prior to dosing.

A “solvate” is intended to mean a pharmaceutically acceptable solvate form of a specified compound that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof.

A “pharmaceutically acceptable salt” is intended to mean a salt that retains the biological effectiveness of the free acids and bases of the specified derivative, containing pharmacologically acceptable anions, and is not biologically or otherwise undesirable. Examples of pharmaceutically acceptable salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, p-toluenesulfonate, tosylate, triethiodode, and valerate salts.

As used herein, “coefficient of variation” or “C.V.” refers to a standard statistical measure of variance and is defined as the standard deviation divided by the mean value. The C.V. can be expressed as a percentage by multiplying by 100.

As used herein with reference to a control composition, the term “consists essentially of Compound A in polymorphic Form IV” is intended to be limited to Compound A in polymorphic Form IV but can also include common excipients that are used in pharmaceutical tablet formulations, but is free from solubilizers or other components that would materially affect the solubility of Compound A.

“Abnormal cell growth”, as used herein, unless otherwise indicated, refers to cell growth that is independent of normal regulatory mechanisms (e.g., loss of contact inhibition), including the abnormal growth of normal cells and the growth of abnormal cells. This includes, but is not limited to, the abnormal growth of: tumor cells (tumors) that proliferate by expressing a mutated tyrosine kinase or overexpression of a receptor tyrosine kinase; benign and malignant cells of other proliferative diseases in which aberrant tyrosine kinase activation occurs; any tumors that proliferate by receptor tyrosine kinases; any tumors that proliferate by aberrant serine/threonine kinase activation; benign and malignant cells of other proliferative diseases in which aberrant serine/threonine kinase activation occurs; tumors, both benign and malignant, expressing an activated Ras oncogene; tumor cells, both benign and malignant, in which the Ras protein is activated as a result of oncogenic mutation in another gene; benign and malignant cells of other proliferative diseases in which aberrant Ras activation occurs. Examples of such benign proliferative diseases are psoriasis, benign prostatic hypertrophy, human papilloma virus (HPV), and restinosis. “Abnormal cell growth” also refers to and includes the abnormal growth of cells, both benign and malignant, resulting from activity of the enzyme farnesyl protein transferase. The terms “abnormal cell growth” and “hyperproliferative disorder” are used interchangeably in this application.

The foregoing and other objectives, features, and advantages of the invention will be more readily understood upon consideration of the following detailed description of the invention.

### BRIEF DESCRIPTION OF THE DRAWINGS

FIG. 1 is an X-ray diffraction pattern of polymorphic Form IV of crystalline Compound A.

FIG. 2 is an X-ray diffraction pattern of the solid amorphous dispersion of Example 2.

### DETAILED DESCRIPTION

Compound A is 6-[2-(methylcarbamoyl)phenylsulfanyl]-3-E-[2-(pyridin-2-yl)ethenyl]indazole and has the following structure:

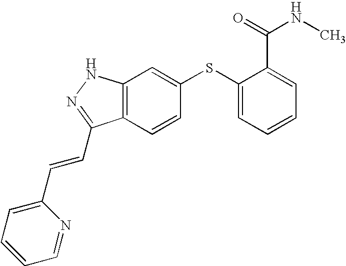

Compound A has beneficial prophylactic and/or therapeutic properties when administered to an animal, especially humans. The term “Compound A” should be understood to include any pharmaceutically acceptable forms of the compound. By “pharmaceutically acceptable forms” is meant any pharmaceutically acceptable derivative or variation, including stereoisomers, stereoisomer mixtures, enantiomers, tautomers, solvates, hydrates, isomorphs, polymorphs, pseudomorphs, neutral forms, salt forms and prodrugs.

Compound A may be synthesized by standard organic synthetic techniques using the procedures outlined in U.S. Pat. No. 6,534,524 (see Example 33(a)), U.S. Provisional Patent Application 60/624,575, filed on Nov. 2, 2004, and U.S. Provisional Patent Application 60/624,635, filed on Nov. 2, 2004, the disclosures of which are all incorporated herein by reference. Since Compound A is a base, pharmaceutically acceptable salts may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

### Amorphous Compound A

In one aspect, a composition comprises amorphous Compound A. By “amorphous” is meant that the compound is not “crystalline.” By “crystalline” is meant that the compound exhibits long-range order in three dimensions. Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC). Preferably, at least a portion of Compound A in the compositions of the present invention is in an amorphous form. Thus, preferably at least 5 wt % of the Compound A in the compositions is in an amorphous form. Generally, the concentration enhancement obtained with amorphous Compound A increases as the amount of Compound A in the composition increases. Thus, the amount of Compound A in an amorphous form may be at least 10 wt %, at least 20 wt %, at least 30 wt %, at least 40 wt %, at least 50 wt %, at least 60 wt %, at least 70 wt %, at least 75 wt %, at least 80 wt %, at least 90 wt %, or even at least 95 wt %. In one embodiment, essentially all of Compound A in the composition is in an amorphous form, meaning that the amount of Compound A in crystalline form is below detection limits using standard quantitative techniques for determining crystallinity in a material.

The inventors have found that amorphous Compound A provides improved concentration of dissolved Compound A in a use environment relative to crystalline Compound A and reduced subject-to-subject pharmacokinetic variability when administered to an in vivo use environment.

### Solid Amorphous Dispersions of Compound A and a Matrix

In one embodiment, a pharmaceutical composition of the present invention comprises a solid amorphous dispersion of Compound A and one or more components, which are collectively referred to as the “matrix.” By “solid amorphous dispersion” is meant that at least a portion of Compound A is in the amorphous form and dispersed in the matrix.

Preferably, at least a portion of Compound A in the compositions of the present invention is in an amorphous form. Thus, preferably at least 5 wt % of the Compound A in the compositions is in an amorphous form. The amount of Compound A in the amorphous form may be at least 10 wt %, at least 20 wt %, at least 30 wt %, at least 40 wt %, at least 50 wt %, at least 60 wt %, at least 70 wt %, at least 75 wt %, at least 80 wt %, at least 90 wt %, or even at least 95 wt %. In one embodiment, essentially all of Compound A in the solid amorphous dispersion is in the amorphous form, meaning that the amount of Compound A in crystalline form is below detection limits using standard quantitative techniques for determining crystallinity in a material.

The amorphous Compound A can exist within the solid amorphous dispersion in relatively pure amorphous domains or regions, as a solid solution of compound homogeneously distributed throughout the matrix or any combination of these states or those states that lie intermediate between them. Preferably, the dispersion is “substantially homogeneous” so that the amorphous compound is dispersed as homogeneously as possible throughout the matrix. As used herein, “substantially homogeneous” means that the compound present in relatively pure amorphous domains within the solid dispersion is relatively small, on the order of less than 20%, and preferably less than 10% of the total amount of Compound A. Dispersions of the present invention that are substantially homogeneous generally are more physically stable and have improved concentration-enhancing properties and, in turn improved bioavailability, relative to non-homogeneous dispersions.

When Compound A and the matrix have glass transition temperatures that differ by more than about 20° C., the fraction of Compound A present in relatively pure amorphous domains or regions within the solid amorphous dispersion can be determined by measuring the glass transition temperature (T_{g}) of the dispersion. T_{g} as used herein is the characteristic temperature at which a glassy material, upon gradual heating, undergoes a relatively rapid (i.e., in 10 to 100 seconds) physical change from a glassy state to a rubbery state. The T_{g} of an amorphous material such as a polymer or dispersion can be measured by several techniques, including by a dynamic mechanical analyzer (DMA), a dilatometer, a dielectric analyzer, and by DSC. The exact values measured by each technique can vary somewhat, but usually fall within 10° to 30° C. of each other. When the solid amorphous dispersion exhibits a single T_{g}, the amount of Compound A in pure amorphous domains or regions in the dispersion is generally less than about 10 wt %, confirming that the dispersion is substantially homogeneous. This is in contrast to a simple physical mixture of particles of pure amorphous Compound A and pure amorphous matrix particles, which generally display two distinct T_{g}s, one being that of Compound A and the other that of the matrix. For a solid amorphous dispersion that exhibits two distinct T_{g}s, it may be concluded that at least a portion of Compound A is present in relatively pure amorphous domains. With DSC, the amount of Compound A present in relatively pure amorphous domains or regions may be determined by first measuring the T_{g} of a substantially homogeneous dispersion with a known amount of Compound A to be used as a calibration standard. From the T_{g} of a homogeneous dispersion, the T_{g} of pure polymer, and the T_{g} of the polymer-rich phase of a dispersion exhibiting two T_{g}s, the fraction of Compound A in relatively pure amorphous domains or regions can be estimated. Alternatively, the amount of Compound A present in relatively pure amorphous domains or regions may be determined by comparing the magnitude of the heat capacity (1) that correlates to the T_{g} of Compound A with (2) that which correlates to the T_{g} of a physical mixture of amorphous Compound A and the matrix.

Preferably, the solid amorphous dispersion exhibits at least one T_{g} intermediate the T_{g} of pure Compound A and the T_{g} of pure matrix material, indicating that at least a portion of Compound A and matrix are present as a solid solution.

The amount of matrix relative to the amount of Compound A present in the dispersion of the present invention depends on the characteristics of the matrix and may vary widely from a Compound A-to-matrix weight ratio of from 0.01 (1 part Compound A to 100 parts matrix) to 100 (i.e., from 1 wt % Compound A to 99 wt % Compound A). Preferably, the Compound A-to-matrix weight ratio ranges from 0.01 to 9 (from 1 wt % Compound A to 90 wt % Compound A), more preferably from 0.05 to 1 (from 5 wt % Compound A to 50 wt % Compound A), and even more preferably from 0.05 to 0.67 (from 5 wt % Compound A to 40 wt % Compound A). When formulated as a solid amorphous dispersion containing 10 wt % Compound A, the inventors have found that bioavailability is increased and subject-to-subject pharmacokinetic variability is reduced relative to the crystalline Compound A alone.

In one embodiment, Compound A and the matrix constitute at least 60 wt % of the total mass of the solid amorphous dispersion. Preferably Compound A and the matrix constitute at least 70 wt %, more preferably at least 80 wt %, and even more preferably at least 90 wt % of the total mass of the solid amorphous dispersion. In another embodiment the solid amorphous dispersion consists essentially of Compound A and the matrix.

Matrix materials suitable for use in the compositions of the present invention should be pharmaceutically acceptable, and should have at least some solubility in aqueous solution at physiologically relevant pHs (e.g. 1 to 8). The components used in the matrix may comprise a mixture of several components. In a preferred embodiment, the matrix is a polymer. Almost any neutral or ionizable polymer that has an aqueous-solubility of at least about 0.1 mg/mL over at least a portion of the pH range of 1 to 8 may be suitable.

When the matrix is a polymer it is preferred that the polymer be “amphiphilic” in nature, meaning that the polymer has hydrophobic and hydrophilic portions. Amphiphilic polymers are preferred because it is believed that such polymers tend to have relatively strong interactions with Compound A and may promote the formation of various types of polymer/compound assemblies in solution. A particularly preferred class of amphiphilic polymers are those that are ionizable, the ionizable portions of such polymers, when ionized, constituting at least a portion of the hydrophilic portions of the polymer. For example, while not wishing to be bound by a particular theory, such polymer/compound assemblies may comprise clusters of Compound A surrounded by the polymer with the polymer's hydrophobic regions turned inward towards the compound and the hydrophilic regions of the polymer turned outward toward the aqueous environment. In the case of ionizable polymers, the hydrophilic regions of the polymer would include the ionized functional groups. In addition, the repulsion of the like charges of the ionized groups of such polymers (where the polymer is ionizable) may serve to limit the size of the polymer/compound assemblies to the nanometer or submicron scale. Such assemblies in solution may well resemble charged polymeric micellar-like structures. In any case, regardless of the mechanism of action, the inventors have observed that such amphiphilic polymers, particularly ionizable cellulosic polymers such as those listed below, have been shown to interact with Compound A so as to maintain a higher concentration of Compound A in an aqueous use environment.

One class of polymers suitable for use with the present invention comprises neutral non-cellulosic polymers. Exemplary polymers include: vinyl polymers and copolymers having at least one substituent selected from the group comprising hydroxyl, alkylacyloxy, and cyclicamido; vinyl copolymers of at least one hydrophilic, hydroxyl-containing repeat unit and at least one hydrophobic, alkyl- or aryl-containing repeat unit; polyvinyl alcohols that have at least a portion of their repeat units in the unhydrolyzed (vinyl acetate) form; polyvinyl alcohol polyvinyl acetate copolymers; polyvinyl pyrrolidone; polyethylene polyvinyl alcohol copolymers, and polyoxyethylene-polyoxypropylene block copolymers (also referred to as poloxamers).

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; high molecular weight proteins such as gelatin and albumin; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers. Exemplary commercial grades of such copolymers include the EUDRAGITS®, which are copolymers of methacrylates and acrylates.

A preferred class of polymers comprises ionizable and neutral (or non-ionizable) cellulosic polymers. By “cellulosic” is meant a cellulose polymer that has been modified by reaction of at least a portion of the hydroxyl groups on the saccharide repeat units with a compound to form an ester or an ether substituent. Preferably, the cellulosic polymer has at least one ester- and/or ether-linked substituent in which the polymer has a degree of substitution of at least 0.05 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate substituents attached via ester linkages to a significant fraction of the cellulosic polymer's hydroxyl groups. Generally, the degree of substitution of each substituent can range from 0.05 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulose polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable cellulosic polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of non-ionizable (neutral) cellulosic polymers is those that are amphiphilic. Exemplary polymers include hydroxypropyl methyl cellulose and hydroxypropyl methyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic substituent may itself be ionizable, such as phthalate or trimellitate substituents.

Exemplary cellulosic polymers that are at least partially ionized at physiologically relevant pHs include: hydroxypropyl methyl cellulose acetate succinate (HPMCAS), hydroxypropyl methyl cellulose succinate, hydroxypropyl cellulose acetate succinate, hydroxyethyl methyl cellulose succinate, hydroxyethyl cellulose acetate succinate, cellulose acetate succinate, methyl cellulose acetate succinate, hydroxypropyl methyl cellulose phthalate (HPMCP), hydroxyethyl methyl cellulose acetate succinate, hydroxyethyl methyl cellulose acetate phthalate, carboxyethyl cellulose, ethylcarboxymethyl cellulose (also referred to as carboxymethylethyl cellulose or CMEC), carboxymethyl cellulose, cellulose acetate phthalate (CAP), methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate (CAT), methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate. The most preferred ionizable cellulosic polymers include hydroxypropyl methyl cellulose acetate succinate, carboxymethyl ethyl cellulose, cellulose acetate phthalate, hydroxypropyl methyl cellulose phthalate, methyl cellulose acetate phthalate, hydroxypropyl cellulose acetate phthalate, hydroxypropyl methyl cellulose acetate phthalate, cellulose acetate trimellitate, hydroxypropyl methyl cellulose acetate trimellitate, cellulose acetate terephthalate, and cellulose acetate isophthalate, and mixtures thereof.

Another preferred class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. Neutralized acidic polymers are described in more detail in the U.S. Published Patent Application US 2003-0054038, entitled “Pharmaceutical Compositions of Drugs and Neutralized Acidic Polymers” filed Jun. 17, 2002, the relevant disclosure of which is incorporated by reference.

While specific polymers have been discussed as being suitable for use in the compositions of the present invention, blends of such polymers may also be suitable. Thus the term “polymer” is intended to include blends of polymers in addition to a single species of polymer.

Of all of these polymers, the most preferred include HPMCAS, HPMCP, HPMC, CAP, CAT, CMEC, poloxamers, and mixtures thereof.

In a preferred embodiment, the matrix is an enteric polymer. By “enteric polymer” is meant a polymer that has an aqueous solubility that is higher at a near neutral pH (pH≧5.5) than at low pH (pH≦5.0). Typically, enteric polymers are relatively insoluble at low pH, typically a pH of less than about 5.0, but at least partially soluble at a pH of greater than about 5.5. The inventors have found that solid amorphous dispersions made using Compound A and an enteric polymer result in reduced pharmacokinetic variability following administration to an in vivo use environment. Without wishing to be bound by any theory or mechanism of action, it is believed that solid amorphous dispersions made with an enteric polymer limit the dissolution rate of Compound A in a gastric environment, where the solubility of Compound A is high. As the composition moves from the low-pH gastric environment to the more neutral pH of the duodenum and intestines, both Compound A and the enteric polymer dissolve in close proximity to each other, resulting in an enhanced concentration of Compound A in the aqueous environment as described above. This results in improved bioavailability of Compound A and a reduced patient-to-patient pharmacokinetic variability. Preferred enteric polymers include HPMCAS, HPMCP, CAP, CAT, CMEC, and mixtures thereof.

In another embodiment, the solid amorphous dispersion comprises Compound A and a blend of an enteric polymer and a low-pH soluble polymer. When administered to a gastric use environment, the low-pH soluble polymer would dissolve with a portion of Compound A. As the composition moves from the low-pH gastric environment to the more neutral pH of the duodenum and intestines, the low-pH soluble polymer inhibits precipitation of Compound A as its solubility decreases. Once in the more neutral pH of the duodenum and small intestines, the enteric polymer and Compound A would dissolve in close proximity to each other, resulting in an enhanced concentration of Compound A in the aqueous environment. Preferred enteric polymers include HPMCAS, HPMCP, CAP, CAT, CMEC, and mixtures thereof. Preferred low-pH soluble polymers include HPMC, hydroxypropyl methyl cellulose acetate, and poloxamers.

In one embodiment, the low-pH soluble polymer is an enteric polymer that is designed to dissolve in an aqueous solution at a pH of 5.5 or less, while the enteric polymer is a polymer that is designed to dissolve in an aqueous solution at a pH of 6.0 or more. An example of an enteric polymer designed to dissolve in an aqueous solution at a pH of 5.5 or less is AQOAT-L made by Shin Etsu (Tokyo, Japan). Examples of enteric polymers designed to dissolve in an aqueous solution at a pH of 6.0 or more include AQOAT-M and AQOAT-H, both available from Shin Etsu.

### Preparation of Solid Amorphous Dispersions

Solid amorphous dispersions comprising Compound A and a matrix may be made according to any conventional process that results in at least a portion of Compound A being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray-coating and spray-drying. Often, processes may form the dispersion by a combination of two or more process types. For example, when an extrusion process is used the extruder may be operated at an elevated temperature such that both mechanical (shear) and thermal (heat) means are used to form the dispersion. Examples of exemplary methods are disclosed in the following U.S. patents, the pertinent disclosures of which are incorporated herein by reference: U.S. Pat. Nos. 5,456,923 and 5,939,099, which describe forming dispersions by extrusion processes; U.S. Pat. Nos. 5,340,591 and 4,673,564, which describe forming dispersions by milling processes; and U.S. Pat. Nos. 5,707,646 and 4,894,235, which describe forming dispersions by melt congeal processes.

A preferred method for forming solid amorphous dispersions of Compound A and a matrix is “solvent processing,” which consists of dissolution of at least a portion of Compound A and at least a portion of the one or more matrix components in a common solvent. The term “solvent” is used broadly and includes mixtures of solvents. “Common” here means that the solvent, which can be a mixture of compounds, will dissolve at least a portion of Compound A and the matrix material(s).

Solvents suitable for solvent processing can be any compound in which Compound A and the matrix are mutually soluble. Preferably, the solvent is also volatile with a boiling point of 150° C. or less. In addition, the solvent should have relatively low toxicity and be removed from the solid amorphous dispersion to a level that is acceptable according to The International Committee on Harmonization (ICH) guidelines. Removal of solvent to this level may require a subsequent processing step such as tray-drying. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, 1,1,1-trichloroethane, and tetrahydrofuran. Lower volatility solvents such as dimethyl acetamide or dimethylsulfoxide can also be used in small amounts in mixtures with a volatile solvent. Mixtures of solvents, such as 50% methanol and 50% acetone, can also be used, as can mixtures with water, so long as the polymer and Compound A are sufficiently soluble to make the spray-drying process practicable. Preferred solvents are methanol, acetone, and mixtures thereof.

After at least a portion of each of Compound A and matrix have been dissolved, the solvent is removed by evaporation or by mixing with a non-solvent. Exemplary processes are spray-drying, spray-coating (pan-coating, fluidized bed coating, etc.), and precipitation by rapid mixing of Compound A and matrix solution with CO₂, hexane, heptane, water of appropriate pH, or some other non-solvent. Preferably, removal of the solvent results in a solid dispersion that is substantially homogeneous. To achieve this end, it is generally desirable to rapidly remove the solvent from the solution such as in a process where the solution is atomized and Compound A and the matrix rapidly solidify.

The solvent may be removed by spray-drying. The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

The amount of Compound A and matrix in the spray solution depends on the solubility of each in the spray solution and the desired ratio of Compound A to matrix in the resulting solid amorphous dispersion. Preferably, the spray solution comprises at least 0.01 wt %, more preferably at least 0.02 wt %, and most preferably at least 0.05 wt % dissolved solids.

The solvent-bearing feed can be spray-dried under a wide variety of conditions and yet still yield solid amorphous dispersions with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of solvent) such that they do not stick to or coat the spray-drying chamber wall.

Although the maximum droplet size varies widely as a function of the size, shape and flow pattern within the spray-dryer, generally droplets should be less than about 500 μm in diameter when they exit the nozzle. Examples of types of nozzles that may be used to form the solid amorphous dispersions include the two-fluid nozzle, the fountain-type nozzle, the flat fan-type nozzle, the pressure nozzle and the rotary atomizer. In a preferred embodiment, a pressure nozzle is used, as disclosed in detail in U.S. Published Patent Application US 2003-0185893, filed Jan. 24, 2003, the disclosure of which is incorporated herein by reference.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas can, in principle, be essentially any gas, but for safety reasons and to minimize undesirable oxidation of Compound A or other materials in the solid amorphous dispersion, an inert gas such as nitrogen, nitrogen-enriched air or argon is utilized. The drying gas is typically introduced into the drying chamber at a temperature between about 60° and about 300° C. and preferably between about 80° and about 240° C. For example, where the spray solution comprises Compound A, HPMCAS, and methanol, the inlet gas temperature may be about 150° C. or less, and more preferably about 135° C. or less.

The large surface-to-volume ratio of the droplets and the large driving force for evaporation of solvent leads to rapid solidification times for the droplets. Solidification times should be less than about 20 seconds, preferably less than about 10 seconds, and more preferably less than 1 second. This rapid solidification is often critical to the particles maintaining a uniform, homogeneous dispersion instead of separating into Compound A-rich and polymer-rich phases. In a preferred embodiment, the height and volume of the spray-dryer are adjusted to provide sufficient time for the droplets to dry prior to impinging on an internal surface of the spray-dryer, as described in detail in U.S. Pat. No. 6,763,607, incorporated herein by reference. As noted above, to obtain large enhancements in concentration and bioavailability it is often necessary to obtain as homogeneous a dispersion as possible.

Following solidification, the solid powder typically stays in the spray-drying chamber for about 5 to 60 seconds, further evaporating solvent from the solid powder. The final solvent content of the solid dispersion as it exits the dryer should be low, since this reduces the mobility of Compound A molecules in the solid amorphous dispersion, thereby improving its stability. Generally, the solvent content of the solid amorphous dispersion as it leaves the spray-drying chamber should be less than 10 wt % and preferably less than 2 wt %.

Following formation, the solid amorphous dispersion can be dried to remove residual solvent using suitable drying processes, such as tray drying, vacuum drying, fluid bed drying, microwave drying, belt drying, rotary drying, and other drying processes known in the art. Preferred secondary drying methods include vacuum drying or tray drying. To minimize chemical degradation during drying, drying may take place under an inert gas such as nitrogen, or may take place under vacuum.

The solid amorphous dispersion is usually in the form of small particles. The mean size of the particles may be less than 500 μm in diameter, or less than 100 μm in diameter, less than 50 μm in diameter or less than 25 μm in diameter. When the solid amorphous dispersion is formed by spray-drying, the resulting dispersion is in the form of such small particles. When the solid amorphous dispersion is formed by other methods such by roto-evaporation, precipitation using a non-solvent, spray-coating, melt-congeal, or extrusion processes, the resulting dispersion may be sieved, ground, or otherwise processed to yield a plurality of small particles.

For ease of processing, the dried particles may have certain density and size characteristics. In one embodiment, the resulting solid amorphous dispersion particles are formed by spray drying and may have a bulk specific volume of less than or equal to about 4 cc/g, and more preferably less than or equal to about 3.5 cc/g. The particles may have a tapped specific volume of less than or equal to about 3 cc/g, and more preferably less than or equal to about 2 cc/g. The particles have a Hausner ratio of less than or equal to 3, and more preferably less than or equal to 2. The particles may have a mean particle diameter up to 150 μm, and more preferably from 1 to 100 μm. The particles may have a Span of less than or equal to 3, and more preferably less than or equal to 2.5. As used herein, “Span,” is defined as

$$Span = \frac{{D}_{90} - {D}_{10}}{{D}_{50}} ,$$

where D₁₀ is the diameter corresponding to the diameter of particles that make up 10% of the total volume containing particles of equal or smaller diameter, D₅₀ is the diameter corresponding to the diameter of particles that make up 50% of the total volume containing particles of equal or smaller diameter, and D₉₀ is the diameter corresponding to the diameter of particles that make up 90% of the total volume containing particles of equal or smaller diameter.

In another embodiment, the solvent is removed by spraying the solvent-bearing feed solution onto seed cores. The seed cores can be made from any suitable material such as starch, microcrystalline cellulose, sugar or wax, by any known method, such as melt- or spray-congealing, extrusion/spheronization, granulation, spray-drying and the like. The feed solution can be sprayed onto such seed cores using coating equipment known in the pharmaceutical arts, such as pan coaters (e.g., Hi-Coater available from Freund Corp. of Tokyo, Japan, Accela-Cota available from Manesty of Liverpool, U.K.), fluidized bed coaters (e.g., Wurster coaters or top-sprayers available from Glatt Air Technologies of Ramsey, N.J. and from Niro Pharma Systems of Bubendorf, Switzerland) and rotary granulators (e.g., CF-Granulator, available from Freund Corp). During this process, the seed cores are coated with the feed solution and the solvent is evaporated, resulting in a coating comprising the solid amorphous dispersion. Forming the solid amorphous dispersion on a seed core has an advantage in that while the dispersion has a low density and thus allows for rapid dissolution when administered to an aqueous use environment, the so-formed particles have an overall density similar to that of the seed core, improving the processing and handling of the composition.

### Concentration Enhancement

In a preferred embodiment, the compositions of the present invention provide concentration enhancement when dosed to an aqueous environment of use, meaning that they meet at least one, and preferably both, of the following conditions. The first condition is that the inventive compositions increase the maximum dissolved concentration (MDC) of Compound A in the environment of use relative to a control composition consisting of an equivalent amount of crystalline Compound A in polymorphic Form IV. That is, once the composition is introduced into an environment of use, the polymer increases the aqueous concentration of Compound A relative to the control composition. It is to be understood that the control composition is free from solubilizers or other components that would materially affect the solubility of Compound A, and that Compound A is in solid form in the control composition. The control composition is crystalline Compound A in polymorphic Form IV, as described in the examples below. Preferably, the inventive compositions provide an MDC of Compound A in aqueous solution that is at least 1.25-fold that provided by the control composition, more preferably at least 2-fold, and most preferably at least 3-fold. Surprisingly, the inventive compositions may achieve extremely large enhancements in aqueous concentration. In some cases, the MDC of Compound A provided by the test composition is at least 5-fold or more that MDC provided by the control.

The second condition is that the inventive compositions increase the dissolution area under the concentration versus time curve (AUC) of Compound A in the environment of use relative to a control composition consisting of an equivalent amount of crystalline Compound A but with no polymer. (The calculation of an AUC is a well-known procedure in the pharmaceutical arts and is described, for example, in Welling, “Pharmacokinetics Processes and Mathematics,” ACS Monograph 185 (1986).) More specifically, in the environment of use, the inventive compositions provide an AUC for any 90-minute period of from 0 to 270 minutes following introduction to the use environment that is at least 1.25-fold that of the control composition described above. Preferably, the AUC provided by the composition is at least 2-fold, more preferably at least 3-fold that of the control composition. Some compositions of the present invention may provide an AUC value that is at least 5-fold, and even more than 10-fold that of a control composition as described above.

As previously mentioned, a “use environment” can be either the in vivo environment, such as the GI tract of an animal, particularly a human, or the in vitro environment of a test solution, such as phosphate buffered saline (PBS) solution or Model Fasted Duodenal (MFD) solution. The inventors have found that in vitro dissolution tests are good predictors of in vivo behavior, and thus compositions are within the scope of the invention if they provide concentration-enhancement in either or both in vitro and in vivo use environments.

The compositions of the present invention provide enhanced concentration of the dissolved Compound A in in vitro dissolution tests. It has been determined that enhanced Compound A concentration in in vitro dissolution tests in MFD solution or in PBS solution is a good indicator of in vivo performance and bioavailability. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. In particular, a composition formed by the inventive method can be dissolution-tested by adding it to MFD or PBS solution and agitating to promote dissolution.

An in vitro test to evaluate enhanced Compound A concentration in aqueous solution can be conducted by (1) adding with agitation a sufficient quantity of control composition, crystalline Compound A, to the in vitro test medium, such as an MFD or a PBS solution, to achieve equilibrium concentration of Compound A; (2) in a separate test, adding with agitation a sufficient quantity of test composition (e.g., the composition comprising Compound A and a matrix) in the same test medium, such that if all Compound A dissolved, the theoretical concentration of Compound A would exceed the equilibrium concentration of crystalline Compound A by a factor of at least 2, and preferably by a factor of at least 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of Compound A dissolved Compound A concentration would be at least 2-fold, preferably at least 10-fold, and most preferably at least 100-fold that of the equilibrium concentration.

The concentration of dissolved Compound A is typically measured as a function of time by sampling the test medium and plotting Compound A concentration in the test medium vs. time so that the MDC can be ascertained. The MDC is taken to be the maximum value of dissolved Compound A measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than 30 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid large particulates of Compound A that would give an erroneous determination, the test solution is either filtered or centrifuged. “Dissolved Compound A” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (≅10-40%) than that obtained with the filter specified above but will still allow identification of preferred dispersions. It should be recognized that this definition of “dissolved Compound A” encompasses not only monomeric solvated Compound A molecules but also a wide range of species such as polymer/Compound A assemblies that have submicron dimensions such as Compound A aggregates, aggregates of mixtures of a matrix and Compound A, micelles, polymeric micelles, colloidal particles or nanocrystals, polymer/Compound A complexes, and other such Compound A-containing species that are present in the filtrate or supernatant in the specified dissolution test.

Alternatively, the compositions, when dosed orally to a human or other animal in the fasted state, provide an AUC in Compound A concentration in the blood (serum or plasma) that is at least 1.25-fold, preferably at least 2-fold, preferably at least 3-fold, preferably at least 5-fold, and even more preferably at least 10-fold that observed when a control composition consisting of an equivalent quantity of crystalline Compound A is dosed. It is noted that such compositions can also be said to have a relative bioavailability of from 1.25-fold to 10-fold that of the control composition.

Alternatively, the compositions, when dosed orally to a human or other animal, provide a maximum dissolved concentration of Compound A in the blood plasma or serum (Cₘₐₓ) that is at least 1.25-fold that observed when a control composition consisting of an equivalent quantity of crystalline Compound A is dosed. Preferably, the blood Cₘₐₓ is at least 2-fold, and more preferably at least 3-fold that of the control composition.

Relative bioavailability of Compound A and the Cₘₐₓ provided by the compositions can be tested in vivo in animals or humans using conventional methods for making such a determination. An in vivo test, such as a crossover study, may be used to determine whether a composition of Compound A and matrix material provides an enhanced relative bioavailability or Cₘₐₓ compared with a control composition as described above. In an in vivo crossover study a test composition of the present invention comprising amorphous Compound A or amorphous Compound A and a matrix is dosed to half a group of test subjects and, after an appropriate washout period (e.g., one week) the same subjects are dosed with a control composition that consists of an equivalent quantity of crystalline Compound A as the test composition. The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC and Cₘₐₓ can be made by plotting the serum or plasma concentration of Compound A along the ordinate (y-axis) against time along the abscissa (x-axis). To facilitate dosing, a dosing vehicle may be used to administer the dose. The dosing vehicle is preferably water, but may also contain materials for suspending the test or control composition, provided these materials do not change the Compound A solubility in vivo.

From in vivo tests, the inventors have found a reduction in subject-to-subject pharmacokinetic variability when Compound A is formulated as a composition of the present invention. By “pharmacokinetic variability” is meant the subject-to-subject variation in AUC and/or Cₘₐₓ in the blood. Subject-to-subject variation can be measured from in vivo determinations of AUC and Cₘₐₓ in the blood using the coefficient of variation (C.V.) over all subjects in the study as a measurement of variability. For example, the AUC C.V. expressed as a percentage, can be determined by dividing the standard deviation of the measured AUC values by the mean AUC value of all measurements, and then multiplying by 100. Preferably, the compositions of the present invention, when administered to a group of at least 4 subjects, provides a C.V. in either the AUC in the blood or Cₘₐₓ in the blood that is 90% or less than the C.V. provided by the control composition. Preferably, the C.V. provided by the compositions of the present invention is 80% or less, and most preferably 70% or less than the C.V. provided by the control composition.

### Dosage Forms

The compositions may be delivered by a wide variety of routes, including, but not limited to, oral, nasal, rectal, vaginal, subcutaneous, intravenous and pulmonary. Generally, the oral route is preferred.

The compositions may also be used in a wide variety of dosage forms for administration of Compound A. Exemplary dosage forms are powders or granules that may be taken orally either dry or reconstituted by addition of water or other liquids to form a paste, slurry, suspension or solution; tablets; capsules; multiparticulates; and pills. Various additives may be mixed, ground, or granulated with the compositions of this invention to form a material suitable for the above dosage forms.

The compositions of the present invention may be formulated in various forms such that they are delivered as a suspension of particles in a liquid vehicle. Such suspensions may be formulated as a liquid or paste at the time of manufacture, or they may be formulated as a dry powder with a liquid, typically water, added at a later time but prior to oral administration. Such powders that are constituted into a suspension are often termed sachets or oral powder for constitution (OPC) formulations. Such dosage forms can be formulated and reconstituted via any known procedure. The simplest approach is to formulate the dosage form as a dry powder that is reconstituted by simply adding water and agitating. Alternatively, the dosage form may be formulated as a liquid and a dry powder that are combined and agitated to form the oral suspension. In yet another embodiment, the dosage form can be formulated as two powders that are reconstituted by first adding water to one powder to form a solution to which the second powder is combined with agitation to form the suspension.

The compositions of the present invention may also be filled into a suitable capsule, such as a hard gelatin capsule or a soft gelatin capsule, well known in the art (see, for example, Remington's The Science and Practice of Pharmacy, 20^{th} Edition, 2000).

In a preferred embodiment, the dosage form is coated with an enteric polymer to limit dissolution of the composition in the stomach. Examples of enteric coatings suitable for this purpose include HPMCAS, HPMCP, CAP, CAT, CMEC, carboxylic acid-functionalized vinyl polymers, such as carboxylic acid functionalized polymethacrylates and carboxylic acid functionalized polyacrylates, and mixtures thereof. Limiting the amount of Compound A that dissolves in the stomach may reduce the patient-to-patient pharmacokinetic variability of Compound A.

### Combination Therapy

The compositions of the present invention may be administered in combination with an additional agent or agents for the treatment of a mammal, such as a human, that is suffering from a disease state associated with abnormal cell growth. The agents that may be used in combination with the compositions of the present invention include, but are not limited to, antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

The invention also relates to a method for the treatment of abnormal cell growth in a mammal which comprises administering to said mammal a therapeutically effective amount of a composition of the present invention in combination with an anti-tumor agent selected from the group consisting of antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

In one embodiment of the present invention the anti-tumor agent used in conjunction with a composition of the present invention is an anti-angiogenesis agent, kinase inhibitor, pan kinase inhibitor or growth factor inhibitor. Preferred pan kinase inhibitors include SU-11248, described in U.S. Pat. No. 6,573,293 (Pfizer Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase 11) inhibitors, MMP-2 (matrix-metalloprotienase 2) inhibitors, and MMP-9 (matrix-metalloprotienase 9) inhibitors. Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron,/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed. Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compositions of the present invention include inhibitors of the enzyme farnesyl protein transferase and inhibitors of the receptor tyrosine kinase PDGFr, including the compounds disclosed and claimed in the following United States patent applications: 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following United States Provisional Patent Applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

PDGRr inhibitors include but are not limited to those disclosed in international patent application publication number WO01/40217, published Jul. 7, 2001 and international patent application publication number WO2004/020431, published Mar. 11, 2004, the contents of which are incorporated in their entirety for all purposes. Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts). GARF inhibitors useful in the practice of the present invention are disclosed in U.S. Pat. No. 5,608,082 which is incorporated in its entirety for all purposes.

Examples of useful COX-II inhibitors which can be used in conjunction with Compound A and pharmaceutical compositions described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381,4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one embodiment the anti-tumor agent is celecoxib as disclosed in U.S. Pat. No. 5,466,823, the contents of which are incorporated by reference in its entirety for all purposes. The structure for Celecoxib is shown below:

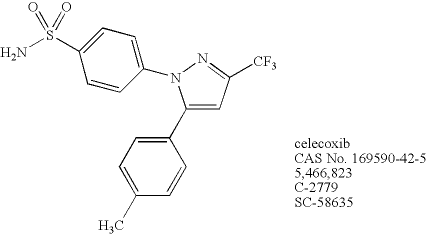

In one embodiment the anti-tumor agent is valecoxib as disclosed in U.S. Pat. No. 5,633,272, the contents of which are incorporated by reference in its entirety for all purposes. The structure for valdecoxib is shown below:

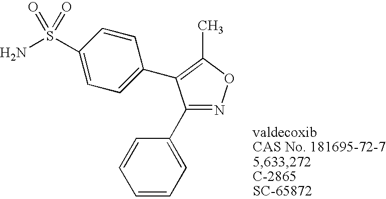

In one embodiment the anti-tumor agent is parecoxib as disclosed in U.S. Pat. No. 5,932,598, the contents of which are incorporated by reference in its entirety for all purposes. The structure for paracoxib is shown below:

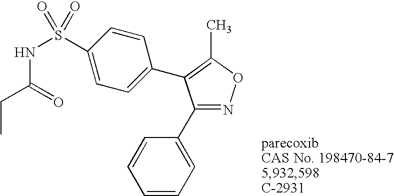

In one embodiment the anti-tumor agent is deracoxib as disclosed in U.S. Pat. No. 5,521,207, the contents of which are incorporated by reference in its entirety for all purposes. The structure for deracoxib is shown below:

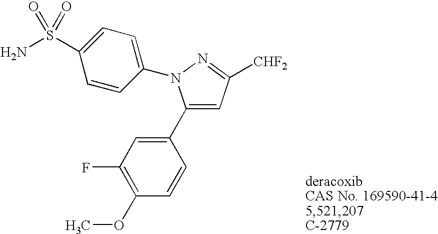

In one embodiment the anti-tumor agent is SD-8381 as disclosed in U.S. Pat. No. 6,034,256, the contents of which are incorporated by reference in its entirety for all purposes. The structure for SD-8381 is shown below:

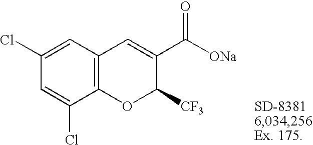

In one embodiment the anti-tumor agent is ABT-963 as disclosed in International Publication Number WO 2002/24719, the contents of which are incorporated by reference in its entirety for all purposes. The structure for ABT-963 is shown below:

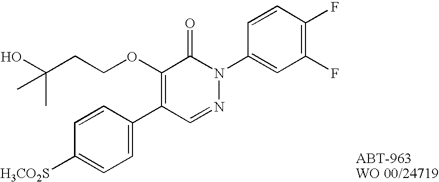

In one embodiment the anti-tumor agent is rofecoxib as shown below:

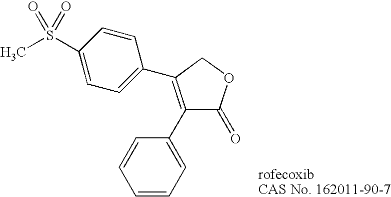

In one embodiment the anti-tumor agent is MK-663 (etoricoxib) as disclosed in International Publication Number WO 1998/03484, the contents of which are incorporated by reference in its entirety for all purposes. The structure for etoricoxib is shown below:

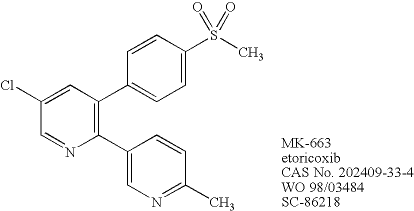

In one embodiment the anti-tumor agent is COX-189 (Lumiracoxib) as disclosed in International Publication Number WO 1999/11605, the contents of which are incorporated by reference in its entirety for all purposes. The structure for Lumiracoxib is shown below:

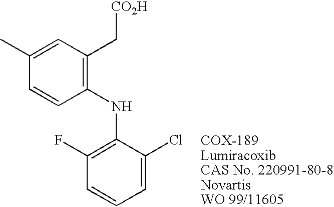

In one embodiment the anti-tumor agent is BMS-347070 as disclosed in U.S. Pat. No. 6,180,651, the contents of which are incorporated by reference in its entirety for all purposes. The structure for BMS-347070 is shown below:

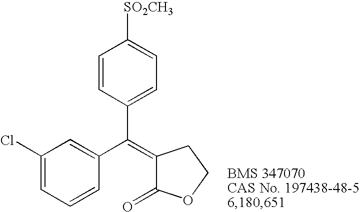

In one embodiment the anti-tumor agent is NS-398 (CAS 123653-11-2). The structure for NS-398 (CAS 123653-11-2) is shown below:

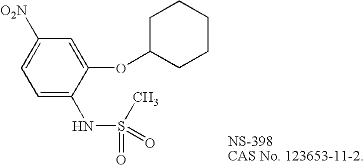

In one embodiment the anti-tumor agent is RS 57067 (CAS 17932-91-3). The structure for RS-57067 (CAS 17932-91-3) is shown below:

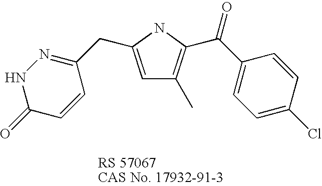

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

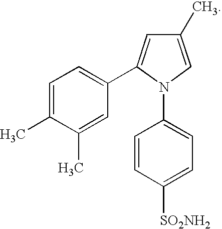

In one embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

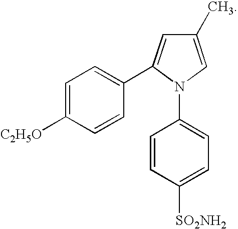

In one embodiment the anti-tumor agent is meloxicam. The structure for meloxicam is shown below:

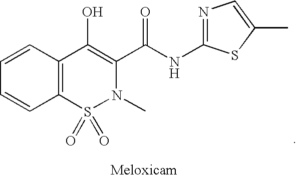

Other useful inhibitors as anti-tumor agents used in conjunction with compositions of the present invention include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-1 inhibitors include ibuprofen (Motrin), nuprin, naproxen (Aleve), indomethacin (Indocin), nabumetone (Relafen) and combinations thereof.

Targeted agents used in conjunction with a composition of the present invention include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Preferred EGFr inhibitors include Iressa, Erbitux, Tarceva and combinations thereof.

Other anti-tumor agents include those selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof. Preferred erb selective anti-tumor agents include Herceptin, TAK-165, CP-724,714, ABX-EGF, HER3 and combinations thereof. Preferred pan erbb receptor inhibitors include GW572016, CI-1033, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof. Further anti-tumor agents may selected from the following agents, CeaVac (CEA), NeuTrexin (trimetresate glucuronate) and combinations thereof. Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof. Additional anti-tumor agents may selected from the following agents, RSR13 (efaproxiral), Cotara (131I chTNT 1/b), NBI-3001 (IL-4) and combinations thereof. Additional anti-tumor agents may selected from the following agents, Canvaxin, GMK vaccine, PEG Interon A, Taxoprexin (DHA/paciltaxel) and combinations thereof. Other anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc./Aton Pharmaceuticals) and combinations thereof. Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in conjunction with a composition of the present invention as described herein.

The invention also contemplates the use of the compositions of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl) propionanilide, bicalutamide) and combinations thereof.

Further, the invention provides a composition of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof. The following topoisomerase I inhibitors may be utilized as anti-tumor agents: camptothecin; irinotecan HCl (Camptosar); edotecarin; orathecin (Supergen); exatecan (Daiichi); BN-80915 (Roche); and combinations thereof. Particularly preferred toposimerase II inhibitors include epirubicin (Ellence).

The compositions of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof. Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, ubenimex and combinations thereof.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof. Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof. Other antitumor agents include mitoxantrone, 1-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additionally, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those described in International Patent Application No. WO 2002/053596, which is herein incorporated by reference in its entirety. Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with a composition of the present invention. Statins (HMG-COA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof. In a preferred embodiment the statin is selected from the group consisting of Atovorstatin and Lovastatin, derivatives and combinations thereof. Other agents useful as anti-tumor agents include Caduet.

Such combinations as described herein may be administered to a mammal such that the compositions of the present invention are present in the same formulation as the additional agents described above. Alternatively, such a combination may be administered to a mammal suffering from a disease state associated with abnormal cell growth such that the compositions of the present invention are present in a formulation that is separate from the formulation in which the additional agent is found. If the compositions of the present invention are administered separately from the additional agent, such administration may take place concomitantly or sequentially with an appropriate period of time in between. The choice of whether to include the compositions of the present invention in the same formulation as the additional agent or agents is within the knowledge of one of ordinary skill in the art.

Other features and embodiments of the invention will become apparent from the following examples that are given for illustration of the invention rather than for limiting its intended scope.

### EXAMPLES

### Synthesis of Crystalline Compound A

A crystalline form of Compound A, designated as polymorphic Form IV, was prepared using the following procedure. Unless otherwise indicated, all temperatures in the following description are in degrees Celsius (° C.) and all parts and percentages are by weight, unless indicated otherwise.

Polymorphic Form IV of Compound A was prepared from a different polymorphic form of Compound A, which is designated as polymorphic Form Ill. Polymorphic Form III of Compound A was prepared by neutralizing a p-toluenesulfonic acid salt derivative of Compound A in ethyl acetate followed by drying under vacuum at 65° C. The p-toluene sulfonic acid salt of Compound A (421 g) was suspended in 1800 mL of 0.84 M NaHCO₃ and 1800 mL ethylacetate and stirred at 65° C. for 2 hrs. Solids were collected by filtration, washed with 1800 mL water and with 800 mL ethylacetate, and dried under lab vacuum at 50° C. overnight to yield the polymorphic Form III of Compound A, which is an ethylacetate solvate. Yield: 92% (HPLC purity was greater than 99%). A sample of polymorphic Form III of Compound A (1.015 kg) was then dissolved in acetic acid at 60° C. The solution was then filtered and concentrated by medium vacuum. 6 L of xylenes were added at 60° C. and then removed by full vacuum. 4 L of xylenes were added and then removed under full vacuum, followed by treatment with an additional 4 L of xylenes. Xylenes were then removed under full vacuum to yield polymorphic Form IV of Compound A in 92% yield. HPLC analysis showed greater than 98.5% purity.

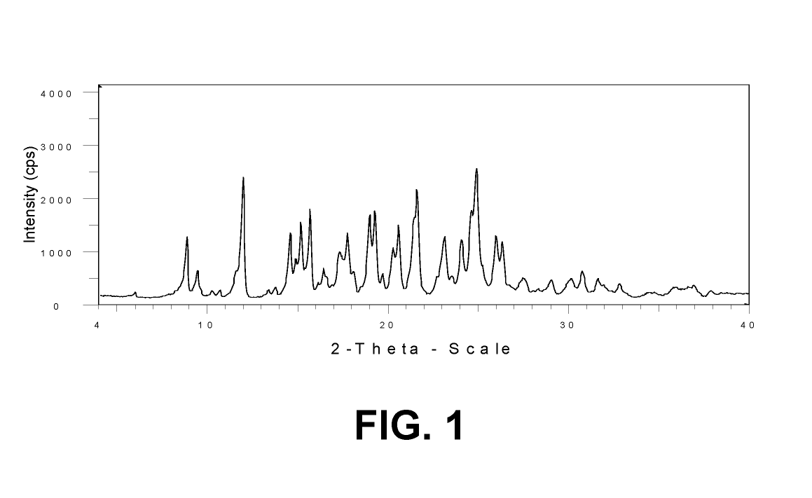

A sample of crystalline Compound A in polymorphic Form IV was examined using powder x-ray diffraction (PXRD) with a Bruker AXS D8 Advance diffractometer. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The x-ray source (KCu_{α}, λ=1.54 Å) was operated at a voltage of 45 kV and a current of 40 mA. Data for each sample were collected over a period of 27 minutes in continuous detector scan mode at a scan speed of 1.8 seconds/step and a step size of 0.04°/step. Diffractograms were collected over the 20 range of 4° to 30°. FIG. 1 gives the PXRD diffractogram of polymorphic Form IV of Compound A.

### Example 1

Amorphous Compound A was prepared from crystalline Compound A by a spray drying process as follows. First, a spray solution was formed by dissolving 100.0 mg crystalline Compound A in polymorphic Form IV and 100 g methanol. The solution was pumped into a “mini” spray-drying apparatus via a Cole Parmer 74900 series rate-controlling syringe pump at a rate of 1.3 mL/min. The compound/polymer solution was atomized through a Spraying Systems Co. two-fluid nozzle, Model No. SU1A using a heated stream of nitrogen at a flow rate of 1 SCFM. The spray solution was sprayed into an 11-cm diameter stainless steel chamber. Heated nitrogen entered the chamber at an inlet temperature of 70° C. and exited at an ambient outlet temperature. The resulting amorphous Compound A was collected on filter paper, dried under vacuum, and stored in a desiccator.

An in vitro dissolution test was performed to determine the dissolution performance of amorphous Compound A relative to crystalline Compound A. For this test, a sufficient amount of material was added to a microcentrifuge test tube so that the concentration of Compound A would have been 200 μgA/mL, if all of the compound had dissolved. The test was run in duplicate. First, 50 μL PBS containing 0.5 wt % Methocel A and 5 mg/mL HPMCAS-H was added to the sample in the tube and mixed using a vortex mixer, to model an oral powder for constitution dosage form. The tubes were placed in a 37° C. temperature-controlled chamber, and 1.8 mL PBS at pH 6.5 and 290 mOsm/kg, containing 7.3 mM sodium taurocholic acid and 1.4 mM of 1-palmitoyl-2-oleyl-sn-glycero-3-phosphocholine, was added to each respective tube. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by HPLC as described above. The contents of each tube were mixed on the vortex mixer and allowed to stand undisturbed at 37° C. until the next sample was taken. Samples were collected at 4, 10, 20, 40, 90, and 1200 minutes.

A similar test was performed with crystalline Compound A alone (Example 1), and a sufficient amount of material was added so that the concentration of compound would have been 200 μgA/mL, if all of the compound had dissolved.

The concentrations of Compound A obtained in these samples were used to determine the maximum dissolved concentration of Compound A (“MDC₉₀”) and the area under the concentration-versus-time curve (“AUC₉₀”) during the initial ninety minutes. The results are shown in Table 1

TABLE 1

| | MDC₉₀ | AUC₉₀ |
|---|---|---|
| Sample | (μgA/mL) | (min * μgA/mL) |
| Example 1 | 48 | 1700 |
| (amorphous Compound A) | | |
| Crystalline Compound A | 3 | 300 |
| (Polymorphic Form IV) | | |

The results show that the amorphous form of Compound A provides concentration-enhancement relative to crystalline Compound A alone. The amorphous form of Compound A provided an MDC₉₀ that was 16-fold that provided by crystalline Compound A, and an AUC₉₀ that was 5.7-fold that provided by crystalline Compound A.

### Example 2

A solid amorphous dispersion containing 10 wt % Compound A and 90 wt % of the “H” grade of hydroxypropyl methyl cellulose acetate succinate (HPMCAS-H, AQOAT-H, available from Shin Etsu, Tokyo, Japan), was prepared as follows. First, a spray solution was formed containing 6.5 g crystalline Compound A, 58.5 g HPMCAS-H, and 8602 g methanol as follows. The crystalline Compound A was added to methanol in a container and stirred for about 2 hours. Next, the HPMCAS-H was added directly to this mixture, and the mixture stirred for an additional 2 hours. The resulting mixture had a slight haze after all the ingredients had been added and dissolved.

The spray solution was added to a tank and pressurized using compressed nitrogen to pass the solution through an inline filter (140 μm screen size) and then to a pressure-swirl atomizer (Schlick #1.5 pressure nozzle) located in a spray-drying chamber.

The spray-drying chamber consisted of three sections: a top section, a straight-side section, and a cone section. The top section was equipped with a drying-gas inlet and a spray-solution inlet. The top section also contained an upper perforated plate and a lower perforated plate for dispersing the drying gas within the spray-drying chamber. The drying gas entered the upper chamber in the top section through the drying-gas inlet, at a flow of about 400 g/min and an inlet temperature of about 135° C. The spray solution was pressurized at a pressure of about 85 psig and fed to the spray-drying chamber through the spray-solution inlet, at a flow rate of about 19 g/min. The pressure-swirl atomizer was mounted flush with the bottom of the lower perforated plate. The spray solution was then sprayed into the straight-side section of the spray-drying chamber. The straight-side section had a diameter of 10.5 inches (26.7 cm) and a length of 31.75 inches (80.6 cm). The flow rate of drying gas and spray solution were selected such that the atomized spray solution was sufficiently dry by the time it reached the walls of the straight-side section that it did not stick to the walls. The spray-dried particles, evaporated solvent, and drying gas exited the spray-drying chamber at a temperature of 57° C., and the spray-dried particles were collected in a cyclone separator.

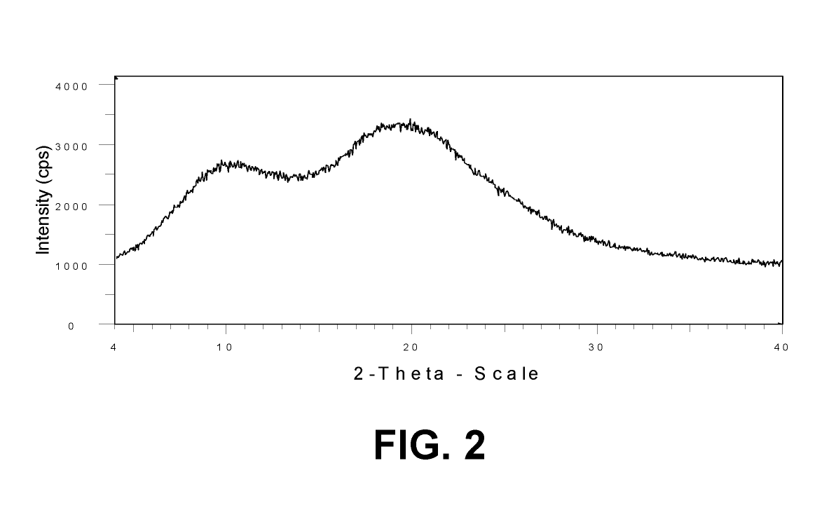

The solid amorphous dispersion formed using the above procedure was post-dried in a vacuum desiccator for 24 hours. The sample was examined using powder x-ray diffraction (PXRD) with a Bruker AXS D8 Advance diffractometer using the procedure outlined above. The results of this analysis, shown in FIG. 2, showed that essentially all of Compound A in the sample was amorphous.

The solid amorphous dispersion was also analyzed using differential scanning calorimetry (DSC). Sample pans were equilibrated at <5% RH, crimped dry, and loaded into a TA Instruments DSC2920. The samples were heated from −60° C. to 225° C. at 2.5° C./min. The glass transition temperature of the sample was determined from the DSC scans, and is shown below in Table 2. The thermal properties for the amorphous Compound A (Example 1), HPMCAS-H, and crystalline Compound A are also included in the table for comparison. The data show that the glass-transition temperature of the solid amorphous dispersion of Example 2 was intermediate that of pure amorphous Compound A (Example 1) and pure polymer, demonstrating that the composition of Example 2 was a homogeneous solid amorphous dispersion.

TABLE 2

| | | Crystalline |
|---|---|---|
| | Glass Transition | Melt |
| | Temperature | Temperature |
| Sample | (° C.) | (° C.) |
| Example 2 | 101 | — |
| (10 wt % Compound A:HPMCAS-H) | | |
| Example 1 | 93 | — |
| (Amorphous Compound A) | | |
| HPMCAS-H | 119 | — |
| Crystalline Compound A | — | 217 |
| (Polymorphic Form IV) | | |

An in vitro dissolution test was performed to determine the dissolution performance of the solid amorphous dispersion of Compound A relative to crystalline Compound A. For this test, a sufficient amount of material was added to a microcentrifuge test tube so that the concentration of Compound A would have been 200 μgA/mL, if all of the compound had dissolved. The test was run in duplicate. The tubes were placed in a 37° C. temperature-controlled chamber, and 1.8 mL PBS at pH 6.5 and 290 mOsm/kg, containing 7.3 mM sodium taurocholic acid and 1.4 mM of 1-palmitoyl-2-oleyl-sn-glycero-3-phosphocholine, was added to each respective tube. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and analyzed by high-performance liquid chromatography (HPLC). HPLC analysis was performed using a Waters C₁₈ column. The mobile phase consisted of 65% 20 mM ammonium phosphate, adjusted to pH 3 with H₃PO₄, and 35% acetonitrile. UV absorbance was measured at 350 nm. The contents of each tube were mixed on the vortex mixer and allowed to stand undisturbed at 37° C. until the next sample was taken. Samples were collected at 4, 10, 20, 40, 90, and 1200 minutes.

A similar test was performed with the crystalline Compound A alone (Example 1), and a sufficient amount of material was added so that the concentration of compound would have been 200 μgA/mL, if all of the compound had dissolved.

The concentrations of Compound A obtained in these samples were used to determine the maximum dissolved concentration of Compound A (“MDC₉₀”) and the area under the concentration-versus-time curve (“AUC₉₀”) during the initial ninety minutes. The results are shown in Table 3.

TABLE 3

| | MDC₉₀ | AUC₉₀ |
|---|---|---|
| Sample | (μgA/mL) | (min * μgA/mL) |
| Example 2 | 60 | 3300 |
| (10 wt % Compound A:HPMCAS-H) | | |
| Crystalline Compound A | 3 | 200 |

The results show that the solid amorphous dispersion of Compound A and HPMCAS-H provides concentration-enhancement relative to crystalline Compound A alone. The solid amorphous dispersion provided an MDC₉₀ that was 20-fold that provided by crystalline Compound A, and an AUC₉₀ that was 16-fold that provided by crystalline Compound A.

A dosage form of the solid amorphous dispersion of Example 2 was made by combining 50 wt % of the solid amorphous dispersion, 15 wt % croscarmellose sodium (AcDiSol, FMC Corp., Philadelphia, Pa.), and 35 wt % microcrystalline cellulose (Avicel PH102, available from FMC Corp.). To form the mixture, the ingredients were weighed and added to a glass container. A stainless steel screen (0.3 cm pore size) was placed in the container, and the ingredients were mixed for 40 minutes using a Turbula mixer. Capsules (#11 porcine gelatin) were filled with 2 g of the blend, for a dose of 100 mg Compound A.

### Examples 3 to 5

Solid amorphous dispersions were prepared using different amounts and types of polymer as indicated in Table 4 using the methods outlined for Example 1, with the exceptions noted in Table 5.

TABLE 4

| | Compound A | |
|---|---|---|
| | Concentration in | |
| Example | Dispersion | |
| No. | (wt %) | Polymer* |
| 3 | 10 | HPMCAS-MG |
| 4 | 25 | HPMCAS-HG |
| 5 | 25 | HPMCAS-MG |

*Polymer designations: HPMCAS-MG = hydroxypropyl methyl cellulose acetate succinate (AQUOT-MG grade, available from Shin Etsu, Tokyo, Japan)

TABLE 5

| | Compound | | Polymer | Solvent |
|---|---|---|---|---|
| Example | A Mass | | Mass | Mass |
| No. | (mg) | Polymer | (mg) | (g) |
| 3 | 50 | HPMCAS-MG | 450 | 50 |
| 4 | 100 | HPMCAS-HG | 300 | 100 |
| 5 | 100 | HPMCAS-MG | 300 | 100 |

Dissolution tests were performed to demonstrate that the solid amorphous dispersions of Examples 3 to 5 provide concentration-enhancement of Compound A. In vitro dissolution tests were performed as in Example 1 (dosed as an oral powder for constitution). For these tests, a sufficient amount of material was added so that the concentration of Compound A would have been 200 μgA/mL, if all of the compound had dissolved. The results are shown in Table 6. The results for the solid amorphous dispersion of Example 2 are included in Table 6, as are the results for tests with pure amorphous Compound A (Example 1) and crystalline Compound A (Example 1).

TABLE 6

| | Compound A | | | |
|---|---|---|---|---|
| | Concentration | | | AUC₉₀ |
| | in Dispersion | | MDC₉₀ | (min * |
| Example | (wt %) | Polymer | (μgA/mL) | μgA/mL) |
| 2 | 10 | HPMCAS-HG | 60 | 3300 |
| 3 | 10 | HPMCAS-MG | 85 | 2300 |
| 4 | 25 | HPMCAS-HG | 103 | 3100 |
| 5 | 25 | HPMCAS-MG | 52 | 2000 |
| 1 | — | — | 48 | 1700 |
| (amorphous | | | | |
| Compound A) | | | | |
| Crystalline | — | — | 3 | 300 |
| Compound A | | | | |
| (Polymorphic | | | | |
| Form IV) | | | | |

These data show that the solid amorphous dispersions of Examples 3 to 5 provided concentration-enhancement over that of the crystalline Compound A alone (Example 1). The solid amorphous dispersions of Examples 3 to 5 provided MDC₉₀ values that were 17-fold to 34-fold that of the crystalline control, and AUC₉₀ values that were 6.7-fold to 10-fold that of the crystalline control.

### Example 6

An in vivo test was performed with male beagle dogs (n=4) in the fasted state using the following protocols. Tablet and spray-dried dispersion compositions (as described in Example 2) of Compound A were administered to male beagle dogs as follows. One group of four fasted male beagle dogs was dosed orally with a spray-dried dispersion composition of Compound A (˜10 mg/kg) on day 1. After a 1 week wash-out period, the same four fasted male beagle dogs were dosed orally with a spray-dried dispersion composition of Compound A (˜0.3 mg/kg) on day 7. Finally on the third week the same four fasted male beagle dogs were dosed orally with a self-emulsifying composition (which is an alternative formulation that is not the subject of the present application, but is described in U.S. Provisional Patent Application entitled “Self-emulsifying Compositions Comprising a VEGF-R Inhibitor”, filed on May 19, 2005) of Compound A (˜0.4 mg/kg). The studies where self-emulsifying and spray-dried dispersion compositions were administered were repeated and mean values reported. The tablet composition was orally administered (8 mg/kg) to the dogs in the fed and fasted states. Dogs in the fasted state were fasted overnight prior to dosing (minimum 12 hours prior to dosing). Blood samples (approximately 0.75 mL) were collected by venipuncture at specified time points (0, 0.25, 0.5, 1, 2, 3, 4, 6, 8, and 24 hours) into tubes containing sodium heparin. Pharmacokinetic data were measured by liquid chromatography-tandem mass spectrometry (LC-MS/MS) and mean values of all studies are shown in Table 7. Data obtained for the self-emulsifying compositions is not shown in Table 7, but can be found in U.S. Provisional Application entitled “Self-emulsifying Compositions Comprising a VEGF-R Inhibitor”, filed on May 19, 2005. In the table, Cₘₐₓ/D is the dose-normalized maximum observed blood plasma concentration of Compound A, AUC_{inf}/D is the dose-normalized AUC, where standard deviations for each are shown in parentheses, and C.V. is the coefficient of variation (standard deviation/mean×100) of AUC_{inf}/D and Cₘₐₓ/D.

Tablets consisting essentially of crystalline Compound A that were used in these studies were prepared as follows. Povidone (4%, w/w) was dissolved in water (5 times, w/w) to form a solution for granulation. Polymorphic Form IV of Compound A, as described in U.S. Provisional Patent Application 60/624,665, filed on Nov. 2, 2004, the disclosure of which is incorporated herein by reference, was combined with lactose (25%, w/w), corn starch (16%, w/w), and a portion of croscarmellose sodium (2%, w/w) in a high sheer granulator. The mixture was dry blended, and then granulated with the povidone solution. The granulation was first wetted for 2 minutes and dried at 60° C. to a loss-on-drying value of 5% or less. The material was then dry milled with screen size 045R. The milled material was blended with the remaining croscarmellose sodium (3%, w/w) and microcrystalline cellulose (12%, w/w). The blended mixture was blended again with magnesium stearate (1%, w/w). Finally, the mixture was compressed using tablet compression equipment to produce tablets.

Capsules containing a solid amorphous dispersion of 10 wt % Compound A in HPMCAS-H were prepared by blending 50 wt % of the dispersion, 15 wt % Ac-Di-Sol, and 35 wt % Avicel PH102 and filling into a gelatin capsule as described in Example 2.

TABLE 7

| | | | | Cₘₐₓ | | AUC |
|---|---|---|---|---|---|---|
| | Fed | Dose | Cₘₐₓ/D (μg/mL/ | C.V. | AUC_{inf}/D | C.V. |
| COMPOSITION | State | mg/kg | mg/kg)) | (%) | (μg * hr/mL/mg/kg) | (%) |
| Crystalline Tablets | Fasted | 8 | 0.051 (0.029) | 57 | 0.19 (0.11) | 58 |
| Crystalline Tablets | Fed | 8 | 0.056 (0.068) | 121 | 0.29 (0.48) | 165 |
| 10% Solid Amorphous | Fasted | 10 | 0.55 (0.197) | 36 | 2.31 (0.85) | 37 |
| Dispersion in Capsule | | | | | | |
| 10% Solid Amorphous | Fasted | 0.3 | 0.47 (0.2) | 43 | 0.93 (0.5) | 54 |
| Dispersion in Capsule | | | | | | |

These results show that the systemic exposure of Compound A increased when delivered in the form of a solid amorphous dispersion as compared to the control composition administered under fasted conditions. The solid amorphous dispersions of the present invention provided a dose-normalized Cₘₐₓ that was 9- to 10-fold that provided by the crystalline Compound A control composition, and a dose-normalized AUC value that was 4- to 12-fold that of the control. The results also show that the solid amorphous dispersion resulted in a significant reduction in pharmacokinetic variability relative to crystalline drug. The Cₘₐₓ C.V. value provided by the 10 mg/kg composition of the present invention was less than 64% of the value provided by the control composition, while the Cₘₐₓ C.V. value provided by the 0.3 mg/kg composition of the present invention was less than 76% of the value provided by the control composition. The AUC C.V. value provided by the 10 mg/kg composition of the present invention was less than 64% of the value provided by the control composition, while the AUC C.V. value provided by the 0.3 mg/kg composition of the present invention was less than 92% of the value provided by the control composition.

All publications, patents, and patent applications cited in this specification are incorporated herein by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A pharmaceutical composition comprising Compound A or a pharmaceutically acceptable salt or solvate thereof, wherein at least a portion of said compound is amorphous.

2. The composition of claim 1 wherein at least 75 wt % of said compound is amorphous.

3. The composition of claim 1 wherein said composition further comprises a matrix.

4. The composition of claim 3 wherein said compound and said matrix are in the form of a solid amorphous dispersion.

5. The composition of claim 3 wherein said matrix is selected from the group consisting of hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, hydroxypropyl methyl cellulose acetate phthalate, cellulose acetate terephthalate and cellulose acetate isophthalate, hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose, and mixtures thereof.

6. The composition of claim 3 wherein said matrix is selected from the group consisting of: carboxylic acid functionalized polymethacrylates; carboxylic acid functionalized polyacrylates; amine-functionalized polyacrylates; amine-fuctionalized polymethacrylates; proteins; carboxylic acid functionalized starches; vinyl polymers and copolymers having at least one substituent selected from the group consisting of hydroxyl, alkylacyloxy, and cyclicamido; vinyl copolymers of at least one hydrophilic, hydroxyl-containing repeat unit and at least one hydrophobic, alkyl- or aryl- containing repeat unit; polyvinyl alcohols that have at least a portion of their repeat units in the unhydrolyzed form; polyvinyl alcohol polyvinyl acetate copolymers; polyethylene glycol polypropylene glycol copolymers; polyvinyl pyrrolidone; polyethylene polyvinyl alcohol copolymers; and polyoxyethylene-polyoxypropylene block copolymers, and mixtures thereof.

7. A pharmaceutical composition comprising Compound A, or a pharmaceutically acceptable salt or solvate thereof, and a matrix, wherein at least a portion of said compound is in an amorphous form, and wherein said composition, when administered to an in vitro aqueous use environment, provides at least one of:
(a) a maximum dissolved concentration of said compound in said use environment that is at least 1.25-fold that provided by a control composition; and
(b) a concentration of said compound in said use environment versus time area under the curve (AUC) for any period of at least 90 minutes between the time of introduction into said use environment and 270 minutes following introduction to said use environment that is at least 1.25-fold that of said control composition;
wherein said control composition consists essentially of an equivalent quantity of said compound in polymorphic Form IV alone.

8. The composition of claim 7 wherein said compound and said matrix are in the form of a solid amorphous dispersion.

9. The composition of claim 7 wherein at least 75 wt % of said compound is amorphous.

10. A pharmaceutical composition comprising Compound A, or a pharmaceutically acceptable salt or solvate thereof, and a matrix, wherein at least a portion of said compound is in an amorphous form, and wherein when administered to an in vivo use environment, said composition provides at least one of:
a) a dose-normalized AUC value of said compound in the blood plasma or serum that is at least 5-fold that provided by a control composition; and
b) a dose-normalized Cₘₐₓ value of said compound in the blood plasma or serum that is at least 5-fold that provided by said control composition;
wherein said control composition is administered under similar conditions as said pharmaceutical composition and consists essentially of Compound A in polymorphic Form IV.

11. A pharmaceutical composition comprising Compound A, or a pharmaceutically acceptable salt or solvate thereof, and a matrix, wherein at least a portion of said compound is in an amorphous form, and wherein when administered to an in vivo use environment in multiple subjects, said composition provides at least one of:
a) an AUC coefficient of variation that is less than 90% of the AUC coefficient of variation provided by a control composition; and
b) a Cₘₐₓ coefficient of variation that is less than 90% of the Cₘₐₓ coefficient of variation provided by said control composition;
wherein said control composition is administered under similar conditions as said pharmaceutical composition and consists essentially of Compound A in polymorphic Form IV.

12. A method of reducing abnormal cell growth in a mammal in need thereof, comprising the step of administering to said mammal a pharmaceutical composition according to claim 1.

13. A process for preparing a pharmaceutical composition comprising:
(a) dissolving a compound in a spray solution comprising a solvent; and
(b) rapidly evaporating said solvent from said spray solution to afford an amorphous form of said compound;
wherein said compound is 6-[2-(methylcarbamoyl)phenylsulfanyl]-3-E-[2-(pyridin-2-yl)ethenyl]indazole, or a pharmaceutically acceptable salt or solvate thereof.

14. The process of claim 13, wherein said spray solution further comprises a matrix.

15. The process of claim 14, wherein said matrix is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, cellulose acetate terephthalate and cellulose acetate isophthalate.
